(19) United States
(12) Patent Application Publication
(10) Pub. No.: US 2025/0034509 A1
(43) Pub. Date: Jan. 30, 2025

(54) APPARATUS AND CORRESPONDING METHOD OF ASCERTAINING AN OPTIMIZED COMPOSITION OF A CULTURE MEDIUM

(71) Applicant: LABMaiTE GmbH, Freiburg (DE)

(72) Inventors: Jonas BERMEITINGER, Schliengen (DE); Dennis RAITH, Stuttgart (DE); Roland MERTELSMANN, Freiburg (DE); Roman MELACHRINOS, Münster (DE); Avani SAPRE, Freiburg (DE); Matias STINGL, Freiburg (DE); Frédéric LAPIERRE, München (DE); Robert HUBER, Oberaudorf (DE)

(73) Assignee: LABMaiTE GmbH, Freiburg (DE)

(21) Appl. No.: 18/783,540

(22) Filed: Jul. 25, 2024

(30) Foreign Application Priority Data
Jul. 25, 2023 (EP) 23187413.2

(51) Int. Cl. C12M 1/36; C12M 1/00; C12M 1/12; C12M 1/26

(52) U.S. Cl. CPC C12M 41/48; C12M 23/58; C12M 33/04; C12M 37/00

(57) ABSTRACT

In order to accelerate the determination of optimal conditions for culturing of a particular biological organism (14), an apparatus (1) and a corresponding method are proposed, with which, with the aid of a computer and autonomously, i.e. without any human involvement, it is possible to determine an optimized composition of a culture medium (3) and, if appropriate, additional optimized active factors that promote culturing of the organism (14). By this approach, it is possible within a very short time to determine the optimized composition of the culture medium (3) for a specific biological organism (14) in an automated manner or, for example, an optimized ratio of at least two organisms (14a, 14b) that are to be cultured together.

### CROSS-REFERENCE TO RELATED APPLICATIONS

This application claims priority from European Patent Application No. 23187413.2, Jul. 25, 2023, which is incorporated herein by reference as if fully set forth.

### TECHNICAL FIELD

The invention relates to an apparatus for computer-implemented determination of an optimized composition of a culture medium and/or an optimized ratio of (at least) two organisms. For this purpose, the apparatus comprises a number of N reaction vessels that are spatially separated, for example by corresponding vessel walls, and are fillable with N compositions of the culture medium. This is because it is possible in this way, in the course of a test series consisting of N culturing tests, where (at least) one biological organism (i.e. in particular the at least two organisms mentioned) is cultured in the respective reaction vessel in each of the N culturing tests, to study the growth/culturing of the organism to be examined (or of the at least two organisms to be examined together) in the respective composition of the culture medium in the respective reaction vessel. In this context, a respective biological process is executed by the (respective) organism (or by at least two organisms) in each of the N reaction vessels, where these processes may differ in their respective extent on account of the different compositions of the respective culture medium and/or a different ratio of the at least two organisms. The optimization of these processes and hence the culturing of the respective organism can be achieved in an autonomous and automated manner by means of the invention.

In addition, the apparatus comprises a robot configured to establish the respective composition of the culture medium in the respective reaction vessel and/or to establish the said ratio of the at least two organisms. In other words, the apparatus can thus establish the respective compositions and/or the said ratio in the respective reaction vessel automatically without any outside human intervention. To wit, it is possible for the respective composition and/or the respective ratio to be examined in the subsequent respective culturing test and evaluated in an automated manner by the apparatus. The robot is thus able, especially alternatively or supplementarily to the variation/establishment of the composition of the culture medium, to adjust the ratio of the at least two organisms in the respective reaction vessel.

In addition, the apparatus preferably comprises at least one process measurement device configured to measure a respective process response or a respective process state which is generated within the respective reaction vessel by the organism during the test series. As will be elucidated in detail hereinafter, the apparatus may preferably also comprise (at least) one separate inoculum bioreactor in which an inoculum (frequently referred to as “pre-culture”, and less commonly also as “inoculate”) of the (respective) organism to be cultured can be stored or is stored. This preculture reactor thus provides, for the robot, a preculture of the (especially respective) organism that is to be cultivated in the reaction vessels in different respective compositions of the culture medium and/or in different ratios together with at least one second organism.

The invention further relates to a corresponding method of computer-implemented determination of an optimized composition of a culture medium (in which at least one organism is to be cultured) and/or of computer-implemented determination of an optimized ratio of at least two organisms that are to be cultured together (in a culture medium). It should be noted even at this point that this method can preferably be executed by means of an apparatus of the invention as claimed and described herein. For example, the production of the culture medium, however, need not necessarily be accomplished with a robot, for instance when the method is used primarily to determine a desired ratio of two organisms for an already optimized (uniform) culture medium.

### BACKGROUND

The culturing of microorganisms in a culture medium in many cases constitutes a challenge to the user, especially when several organisms, for example co-cultures, are to be cultured together in the culture medium. If a particular model organism is to be studied in detail, it is often necessary first to start laborious and complex preliminary tests in order to discover a suitable culture medium at all and at the same time also to vary the composition of the constituents of this culture medium until the model organism (or if appropriate the organisms) can grow or simply be kept alive in the culture medium even over prolonged periods, where the model organism may also possibly produce and/or consume/metabolize a substance.

It is likewise nontrivial in the case of co-cultures to establish a suitable ratio of the organisms involved in order that these can be successfully cultured together, even when a suitable culture medium is already known. But here too, specifically the choice of composition of the culture medium can have a crucial effect on the culturing of the organisms.

In the prior art, conventional methods, for example “one-factor-at-a-time”, have been used to date, which often allow causal relationships between the model organism and the corresponding culture medium to be ascertained, and then, on the basis of those relationships, a model to be created that allows optimization of the culture medium.

In addition, a further conventional approach is to conduct statistical test plans, called design-of-experiments (DoE), in which several active parameters are often varied simultaneously in order thus rapidly and quickly to find an optimized composition of a culture medium or a suitable ratio of two organisms. In this approach, however, it is often no longer possible to ascertain causal relationships, especially when they are too complex.

### SUMMARY

Proceeding from this known prior art and prior publications WO 2020/016223 A1 and EP 4 039 791 A1, the problem addressed by the invention was that of developing a new approach with which the labour and time that have to be expended for culturing of organisms can be reduced.

This object is achieved in accordance with the invention, in the case of an apparatus, by provision of one or more of the features disclosed herein. In particular, it is thus proposed in accordance with the invention for achievement of the object, in an apparatus of the type specified at the outset, that the apparatus has a controller configured to assess the total of N process responses and/or N process states that have been measured by means of the at least one process measurement device during the respective test series, in each case in a computer-implemented manner with reference to a common objective which is applicable to all the processes that proceed in the reaction vessels in parallel, preferably simultaneously. It is further the case that the controller is configured, on the basis of those N assessments, in a computer-implemented manner, to give the following information to the robot in the form of an instruction: the respective composition of the culture medium which is to be established in the respective reaction vessel in a subsequent test series. Subsequently, the robot, at the start of the subsequent next test series, can make up the defined composition of the culture medium in the respective reaction vessel.

Such a measured process state may, for example, be a particular current biomass concentration or a particular pH or some other measurable state parameter that characterizes the current state of the organism (for example at the end of a culturing test) that is being cultured at any time in the respective reaction vessel. A measured process response, for instance the change in biomass concentration or an altered spectrum attributable to a biological process being executed by the organism, may enable conclusions, for example, as to a particular activity of the organism, for example a metabolism or a particular substance production or substance consumption. For instance, the apparatus can autonomously optimize the composition of the culture medium with regard to a desired rate of substance production or substance consumption (by the organism).

Alternatively or else supplementarily to the latter feature, it may be the case that the controller is configured to specify, on the basis of the N assessments, a ratio of at least two different organisms (that are being cultured together in the respective reaction vessel during a culturing test), which ratio is to be established in the respective reaction vessel in a subsequent test series, to the robot in the form of an instruction. This instruction thus stipulates the relative ratio in which the at least two different organisms are to be re-examined in the subsequent test series. In this case, for example, a composition of the culture medium that has already been optimized beforehand (especially with the aid of the method of the invention) can be retained, or this composition is still to be optimized only at a later stage, or else it is optimized simultaneously with the ratio of the organisms by the controller. Subsequently, the robot can then, at the start of the subsequent new test series, set the defined ratio of the at least two organisms in the respective reaction vessel.

It is thus possible by means of the invention not just to conduct culture medium optimization but, for example, also optimization of induction of a particular protein expression or, for example, optimization of particular antibiotic combinations. This results in numerous possible applications of the invention in different fields of bioprocess technology, medical technology and pharmaceutical process technology.

A significant advantage of the invention is that it enables the apparatus of the invention to ascertain an optimal composition of a culture medium in an automated manner and without human involvement (i.e. autonomously), for a very specific organism (human or animal cell, bacterium, fungus etc.) or else for a particular combination of at least two organisms. This concept is applicable not only to bacteria but also to other microorganisms, and to plant, animal or human cells that are to be cultured, and additionally also, for example, to co-cultures. In the long term, this could especially give rise to medical applications because specifically the culturing of cells is still a major challenge in respect of patient-specific therapy, and this can be addressed by the approach of the invention.

The invention also appears to be of high interest for bioprocess technology, since it is possible with the aid of the invention to very rapidly ascertain, in a cost- and time-optimized manner, how a culture medium can be optimized to a particular biological organism.

However, the apparatus of the invention (and the corresponding method which will be elucidated later on) cannot just be used to examine a single organism. Instead, in one possible configuration, it may be the case that at least two different biological organisms (especially what are called co-cultures or synthetic microbial consortia) are cultured in individual or all of the N reaction vessels during a test series/during an individual culturing test. In this context, the controller may be configured to define, preferably with the aid of the robot (or else in some other way), in a computer-implemented manner, a ratio (especially a volume/mass ratio or mixing ratio) of the at least two different biological organisms in the respective reaction vessel on the basis of the N assessments elucidated above (which are then each made for a process attributable to individual or else all of the at least two organisms).

In this case, it is even possible that the controller, in a test series, first always uses the same composition of the culture medium, in order first to ascertain an optimal ratio of the at least two different organisms for that composition. By controlled mixing of the at least two different organisms, it is possible, for example, to optimize formation of a particular product (as one possible target parameter/objective observed by the controller) in which the organisms are involved in a fully automated manner with the apparatus. When multiple different organisms are used, there is an increase in the complexity of the solution space, but the approach of the invention, through the use of artificial intelligence, unlike in the case of conventional methods, is capable of achieving optimization even in that case.

It is thus also possible to use apparatus of the invention in order to examine the interaction of multiple microorganisms in a particular culture medium (which is always the same under some circumstances). In this case, the controller, especially on the basis of artificial intelligence, can additionally or else alternatively to the composition of the culture medium define the ratio of the at least two different biological organisms in the respective reaction vessel. If, for example, three, four or even more different organisms are examined with the apparatus, the controller may—similarly to the case of the different constituents of the culture medium—also completely omit individual organisms among the different organisms. For instance, the apparatus/artificial intelligence can especially make a selection among different organisms that are stored in the apparatus in order to achieve a particular objective.

The apparatus can adjust the ratio of the at least two different biological organisms, for example, in that, at the start of a test series, a particular ratio of amounts or concentrations of at least two different inocula (each comprising one or more of the respective at least two different organisms) is established in the respective reaction vessel with the robot. It is not absolutely necessary here to adjust the biomass concentration in the respectively stored inoculum; instead, different amounts of the inoculum may be dispensed into the respective reaction vessel and/or, for example, a sample of the appropriate inoculum may be correspondingly diluted in order thus to lower the biomass concentration of the organism in that sample.

Accordingly, the apparatus may also have at least two inoculum bioreactors (these may be implemented in one piece of equipment). As a result, it is possible, for example, for one inoculum to be storable or stored for each/one/more than one of the at least two different organisms.

The use of spatially separate reaction vessels has the advantage that the biological processes can proceed simultaneously but spatially separately in the respective reaction vessel and hence unaffected by one another, such that different compositions of the culture medium and/or different ratios of the different organisms can be examined simultaneously by the apparatus (with regard to a desired process aim, for example significant and/or rapid growth of the organism and/or desired substance production/substance consumption by the organism, i.e. in particular a desired metabolism of the organism). This parallel and automated approach achieves enormous acceleration of the examination, such that the apparatus is able to determine the optimized composition of the culture medium within a few days to weeks and possibly additionally further optimized culture conditions (such as optimized values for additional active parameters to be established, for instance temperature of the reaction vessel, the radiation intensity, composition of a gas atmosphere in the reaction vessel, etc.), or, for example, an optimized ratio of at least two different organisms that are intended to interact in a particular composition of the culture medium (according to the objective).

The culture medium, which may be configured, for example, in the form of a nutrient solution, thus comprises several media or constituents that are important for the growth of the organism (the organisms in some cases) and/or its metabolism (their metabolism in some cases). By adjustment of the volume ratios of these media within the respective reaction vessel, the robot is able to establish different compositions of the culture medium in the respective reaction vessel; in this case, under some circumstances, the controller instruction can go to such an extent that individual constituents of the culture medium are no longer released at all into the respective reaction vessel. In other words, the exact composition of the culture medium in relation to ingredients and its (mass/volume) ratios may vary in the course of a test sequence performed by the apparatus (which may comprise one or more test series). In the extreme case, the apparatus can start the test sequence with a first composition and present a composition of the culture medium as optimum at its end, which differs fundamentally from the culture medium initially used. It should also be mentioned that the number N of culturing tests running in parallel at any time can change, especially decrease, in the course of a test sequence.

When the robot executes the instruction, it establishes a respective composition of the culture medium which is fixed in the instruction in each of the N reaction vessels as a starting condition for a new test series of N culturing tests and/or it provides a particular ratio (based on the biomass concentration) between the at least two organisms in the respective reaction vessel. The instruction thus comprises at least one data set of N compositions and/or ratios that are especially (but not necessarily) different.

This apparatus of the invention can especially be used in order to implement computer-implemented control of a multitude of biological processes (that proceed in the individual reaction vessels, especially simultaneously), in order thus to examine and to optimize, in an automated manner, the culturing of an organism to be examined in the culture medium. This is because, by means of the composition of the culture medium defined with the aid of the robot in which the organism grows or is cultured in each case during the respective test series (i.e., for example, continuously performs at least one desired metabolic process), the growth conditions and/or life conditions can be set specifically as active parameter and hence the biological process can be controlled. It is likewise possible to consider a ratio of the at least two organisms to be an active parameter, since this may also be crucial for the growth conditions and/or life conditions of the respective organisms and/or for a process aim to be achieved, for example a particular metabolism.

The culture medium that serves for culturing of the organism to be examined can thus be set specifically by the apparatus in order to achieve a particular desired growth process of the organism or else in order at least to maintain a particular desired metabolic activity of the organism (possibly without any major growth or without any growth of the organism). The invention is thus not limited to computer-implemented control/regulation of biological growth processes, but can be used quite generally to control a biological process executed by the organism (on the basis of the culture medium established by the apparatus). This process may also consist in coexistence of at least two organisms in which all these organisms are cultured successfully (for example in a symbiosis).

In this context, the apparatus (more specifically the controller) is able also to adjust further active parameters, for instance one global temperature or respective temperatures of the individual reaction vessels and/or a global and/or respective irradiation of the reaction vessel with light (for example in the case of a photosynthesizing organism) and/or a global or else respective composition of a gas atmosphere in the reaction vessel, on the basis of the respective assessment, made in a computer-implemented manner, of the measured process response, i.e. to define them in adapted (optimized) form for the subsequent test series. “Globally” here may mean that the respective active parameter is adjusted collectively for all N reaction vessels, for instance in that a gas atmosphere in which the reaction vessels are present is adjusted collectively for all vessels.

The set of instructions may thus comprise at least one further active parameter (temperature of the reaction vessel or irradiation intensity to be applied or composition of a gas atmosphere) that is to be (and is) defined (individually) by the apparatus during the running test series for each of the N reaction vessels. The set of instructions thus comprises at least N different compositions of the culture medium that are to be made up in each case by the robot for the new test series in the N reaction vessels.

It may also be the case, for example, that individual compositions among the N compositions are the same but a different active parameter, for example the temperature of that reaction vessel in which that composition is being used for culturing of the organism at any time or the said ratio of the at least two organisms, is varied. Such a variation of a further active parameter, for instance the temperature or said ratio, may then thus exist between at least two reaction vessels having the same composition of the culture medium (at least during the culturing test proceeding at that time).

By virtue of the approach of the invention, the apparatus, more specifically the controller, can thus learn independently how the organism(s) can be optimally cultured in the culture medium. The ascertained optimized composition of the culture medium and/or the optimized active parameters (for instance an optimized ratio of two organisms) can then be provided by the apparatus at the end of the test sequence, preferably in the form of a digital dataset.

In this context, the controller is able to especially itself decide on the basis of assessments how many test series are conducted successively (within the overall test sequence of multiple test series conducted in succession). The controller is thus able to independently determine the stoppage of the test sequence and hence the duration required for the overall test sequence.

The objective defines a desired process response which is to be achieved in culturing of the organism in the respective culture medium, for example a particular growth of biomass of the organism (by replication of the organism itself) or a particular growth rate (growth of biomass per time interval) or a particular substance production/time or a particular substance consumption/time that the organism is to achieve, or a particular state of the organism that is to be maintained for a particularly long period of time. Since the optimal composition of the culture medium, however, is unknown at first, the apparatus can at first randomly select the compositions of the culture medium for a first series of N culturing tests that run in parallel in the N individual reaction vessels (i.e. the organism is cultured in the respective culture medium within the respective reaction vessel), i.e. fix random starting conditions initially (i.e. in the first test series of the sequence). The same also applies to the ratio of the at least two organisms.

On the basis of the measured process responses/process states in this first series of culturing tests, the apparatus, or more specifically the controller, is then able to define the starting conditions for a subsequent series of N culturing tests that are conducted in an automated manner by the apparatus with the same organism in the same N reaction vessels. For this purpose, the controller is able to transmit a corresponding set of respective N instructions (these may be summarized in one, especially digital, instruction) to the robot for production of N adjusted compositions of the culture medium, in order that the robot establishes these N adjusted compositions of the culture medium in the individual reaction vessels.

A suitable target parameter that may be used as objective in the context of the invention may be defined, for example, as a particular level or a particular growth of biomass.

The growth of organisms often proceeds like an S curve. Therefore, the objective on the basis of which the controller/Al assesses the respective process response/process state may correspond, for example, to a plateau to be attained in this S curve or else, for example, a maximum slope in the S curve; in other words, the objective may be, for example, a particular biomass concentration (measured, for example, as a volume concentration of the organism within the reaction vessel/culture medium) or a particular growth rate to be achieved (determined, for example, from the measurements of process response as growth of biomass in grams per unit time and volume). In addition, the objective may also be a specific growth rate u (typically for a monoculture), especially a progression of growth rate u versus time. This is because such a measurement parameter is of excellent suitability for control of microbiological processes. Therefore, the growth rate u is indeed frequently measured in bioprocess technology. If this is done using a flow cytometer, for example, in the process measurement device, it may be possible also to detect, as two process responses, the respective individual growth rates of two organisms being cultured simultaneously in one of the reaction vessels.

The measurement parameter which is measured by means of the process measurement device may preferably be an optical density (for example in order to achieve an optical measurement of turbidity) and/or an intensity value of backscattered light (backscattered by the culture medium and the organism). Optical density or absorbance is a measure of the attenuation of radiation after passing through a medium. The culture medium here may preferably be created/chosen such that it does not itself show any significant turbidity/optical absorption or backscatter of the light used in the measurement. For example, an optical measurement signal may arise only after introduction of an organism and subsequent growth of that organism, for instance because the organism makes the culture medium increasingly cloudy and/or causes optical backscatter.

It is assumed here that the higher the measured optical turbidity and/or the higher the intensity of the backscattered light, the higher the biomass concentration in the respective reaction vessel. Such an optical assessment of the change in biomass concentration in the respective reaction vessel (as a possible process response which is generated by the organism itself) allows comparison, for example, of a particular desired growth/time, i.e. a target growth rate, as objective with this measured process response in order to assess the latter (the assessment could be, for example: “growth rate too high” or “growth rate too low”).

On the basis of such a respective assessment, the controller, especially by means of artificial intelligence, as will be described in more detail, is able to assess whether the respective composition is favourable in order to bring the process response and/or process state closer to the objective, or more unfavourable. Accordingly, it is then possible to examine the favourable compositions in a subsequent test series, for instance in which further adjustments to the culture medium, which may be smaller or additional in some cases, are undertaken by the apparatus in the respective reaction vessel. In this case, the number N of culturing tests that proceed in parallel may of course also decrease, for example if only a sub-selection is to be examined in detail, or else increase, for instance if finer gradations of the adjustments made are required in the respective culturing test.

As will be elucidated in detail, it is advantageous when the reaction vessels are cleaned and disinfected by the apparatus between the individual test series (each test series consists of N individual culturing tests that proceed in parallel) which form a test sequence, such that, in the case of production of the adjusted compositions, controlled starting conditions exist again for the next test series.

Moreover, each of the reaction vessels may be newly inoculated with the organism to start the respective culturing test in that a defined amount of an inoculum of the organism, with known volume concentration of the organism, is introduced into the respective reaction vessel by the robot. These N inoculations of the N reaction vessels at the start of each new test series can thus likewise be conducted in a fully automated manner.

The reaction vessels may be implemented, for example, by the individual microtitre cavities (wells, especially microwells) of a microtitre plate (microwell plate). In other words, it is thus possible to use, in particular, spatially separated, semi-open reaction vessels.

It is possible here for the individual reaction vessels to have volumes of a few ml. As a result, the apparatus may have a compact configuration, and a high throughput may simultaneously be achieved because numerous reaction vessels (for example at least 6, at least 48 or even at least 96 reaction vessels, especially when multiple well plates/bioreactors are used) can be used simultaneously. The apparatus can thus achieve automated high-throughput screening (HTS) of different compositions of a culture medium to be examined, in which case the same organism can always be cultured in the different compositions, or else, as mentioned, for example, two or more different organisms. Each of the reaction vessels is thus able to provide, after filling with the culture medium, a reaction volume in which the organism can execute the desired biological process (growth of the organism itself or a particular metabolic process attributed to the organism).

The reaction vessels in which the culturing tests take place may alternatively also be configured in a bioreactor, in particular in a bioreactor block (for example bioREACTOR 48 from 2 mag AG). It is then possible here for each reaction vessel to be formed by a closed mini-reactor. By this approach, the invention can be used, for example, for the optimization of high-throughput culturing (i.e., for example, microbial transformation of organic substances by probiotic bacteria and/or fungi) in the fields of biotechnology, chemistry and pharmacy. Such bioreactors can also be operated in a fully automated manner with the aid of the pipetting robot of the apparatus of the invention. And miniaturization (volume of the reaction vessels of a few ml) also contributes here to a significant saving of materials and costs.

In the case of such bioreactors, for example, it is possible to achieve automatic speed monitoring of a respective stirrer bar and non-invasive real-time measurement of pH and dissolved oxygen (DO), and likewise sparging and mixing of the individual reaction vessels, for example by means of gas-inducing inductively driven magnetic stirrer units. It will be apparent that such measurement or control parameters are possible active parameters that can likewise be detected and/or defined by the apparatus of the invention, more specifically the controller, in a computer-implemented manner during a test series. The controller may thus be configured to define, on the basis of the N assessments, at least one active parameter of a bioreactor in a computer-implemented manner (where the bioreactor provides the N reaction vessels) and/or detect at least one measurement parameter of this bioreactor in an automated manner. It is of course also possible in such an apparatus of the invention to operate multiple such bioreactors in parallel and/or to control them centrally by the controller.

It will be apparent that the apparatus may have multiple media vessels in which constituents, referred to as media hereinafter, of the culture medium are storable. As a result, the apparatus, more specifically said robot, is able to assemble respective different compositions of the culture medium from these media, especially within the respective reaction vessel.

Finally, the apparatus may preferably also have a filter module or other module with which an atmosphere in which the reaction vessels are present can be controlled. For example, it is possible to implement sterile headspace gassing above the reaction vessels/the bioreactor for instance in order to prevent cross-contamination and outside contamination. For example, it is possible to culture aerobic and anaerobic microorganisms alike in the apparatus.

The object can also be achieved in accordance with the invention by further advantageous executions as described below and in the claims, which will be elucidated in detail hereinafter:

As mentioned, the robot may preferably be configured as a pipetting robot, i.e., for example, with an electronically actuatable pipette, with which liquids can be taken up and dispensed (in an automated/electronically controlled manner). In particular, the pipetting robot may have a movable pipetting arm configured to be positioned above any one of the total of N reaction vessels. Such a configuration permits dispensing of individual media/components of the culture medium into the respective reaction vessel and/or sucking of the respective culture medium out of the respective reaction vessel with the aid of the pipetting robot. Alternatively, for this purpose, however, the robot may also be configured, for example, without a pipette, for instance with the aid of a dispensing and/or suction hose.

It is preferably the case here that the apparatus includes a sterilizing means for automated sterilization of a pipette tip or a dispensing hose of the pipetting robot. This sterilizing means may be implemented, for example, by means of a cleaning station in which the pipetting robot can clean the pipette tip from time to time, such that the pipette tip can be kept sterile in an automated manner. In this way, it is possible, for example, to effectively prevent cross-contaminations between the individual reaction vessels that would seriously disrupt the test series.

It may be the case here that the robot can draw the individual constituents/components/media of the culture medium from reservoir vessels of the apparatus and pipette/dispense them into the respective reaction vessel in an exactly metered amount, in order thus to establish a particular composition of the culture medium in the reaction vessel.

In a particularly preferred configuration, the controller controls the robot by means of artificial intelligence. This may especially be implemented in that the apparatus learns autonomously from the measured process responses and/or from the measured process states over the course of multiple test series conducted in succession how the robot can establish an optimized composition of the culture medium.

Artificial intelligence (AI) can also take account of at least one non-technical boundary condition, for example the cost of individual constituents of the culture medium. In other words, the controller/the Al can optimize the composition of the culture medium to be defined and/or the respective proportions of the different organisms using multiple criteria, especially the resulting cost of the culture medium, in the course of the test sequence. This is possible if the non-technical boundary conditions are taken into account by the controller as part of the objective (Minimize cost! Prefer organism X!) in the assessment of the process response (and hence also of its underlying composition of the culture medium).

What exactly an optimized composition of the culture medium is can thus be decided by the controller on the basis of the measured process response and, if appropriate, on the basis of further boundary conditions: the closer the apparatus has brought the process response to the objective (by appropriate adjustment of the composition and, if appropriate, of further active parameters, i.e. active factors, that additionally influence the culturing of the organism in this composition of the culture medium), the greater the degree to which the composition has approached an optimum. If no significant improvement in process response is achieved in subsequent test series, the controller can decide independently to stop the test sequence or, for example, still to experiment as to whether similar good results are achievable with a different composition optimized in relation to a non-technical boundary condition. For this purpose, for example, a particular stoppage criterion may be fixed in advance in relation to a delta between that of the N measured process responses which is closest to the objective and the objective itself.

As already mentioned, the controller may have artificial intelligence (AI) which may especially be based on machine learning or implements a machine learning algorithm. With the aid of this artificial intelligence, the controller is able to learn independently, i.e. in particular without human involvement, an optimized composition of the culture medium from the respectively measured process responses and to output this optimized composition (for example in the form of a digital dataset), for example at the end of a sequence of multiple test series (successive in time) that have been conducted with the apparatus.

The respective composition of the culture medium envisaged for one of the reaction vessels (especially in the course of a single culturing test within a test sequence being conducted by the apparatus) can thus be adjusted by means of artificial intelligence (AI) in a computer-implemented manner. For this purpose, it is possible in particular to use, i.e. for the controller to implement, machine learning methods, for example a deep learning method and/or artificial neural networks (ANNs).

Among the features of the approach pursued by the invention are thus that artificial intelligence, for example with the aid of machine learning, learns autonomously how the optimal composition of the culture medium should be in order to achieve the desired process response with the organism. In this context, however, the Al specifically does not need to understand the underlying causal relationships, i.e. the biochemical interactions between culture medium and organism; instead, the Al learns from empirical observations, namely the process responses measured in the test series.

As already mentioned at the outset, the apparatus may preferably comprise an inoculum bioreactor. To wit, an inoculum of the organism to be cultivated may be stored or is stored in this bioreactor. In this case, the robot may especially be configured to take a sample of the inoculum from the inoculum bioreactor and to inoculate the respective reaction vessel with that sample, which can typically be implemented at the start of a test series.

An inoculum (or else pre-culture) generally means an inoculation culture, i.e. a particular amount of a pure culture of a (micro) organism that can be used for (further) culturing. In the context of the invention, inoculum may especially mean an inoculation culture which, as well as the biological organism to be cultured, also comprises (inoculum) culture medium in which the organism grows or is culturable. In this context, the culture medium of the inoculum may be chosen differently from that culture medium whose composition is to be specifically optimized by the apparatus.

The invention has recognized in particular that mutations can typically occur in many biological organisms when they are cultured over prolonged periods of time (especially as an inoculum). However, because of the high speed which can be achieved with the apparatus of the invention in the performance of the test sequence, the total time required to determine an optimized composition of the culture medium with the aid of Al is only a few days to a few weeks, depending on the organism. Experience has shown, however, that no relevant mutations occur within such periods of time, and so this specifically does not hinder this approach of the invention.

In order to prevent contamination of the inoculum with other organisms, the taking of the sample can especially be conducted through a sterile barrier (for example in the form of a membrane) that protects an inoculum reaction vessel of the inoculum bioreactor from outside influences. In the same way, it is preferably also possible to protect the reaction vessels in which the culturing tests are conducted from outside influences by means of a sterile barrier. In addition, as explained, it is also possible for the respective composition of an atmosphere in which the respective reaction vessel is present to be controlled, for instance with an HEPA module.

For example, the robot may be configured to take a particular maximal amount as a sample, for example not more than 5% by volume, of the inoculum stored in the bioreactor and distribute this sample between the N reaction vessels, in an exactly metered and equal amount in each case. While the test series is running, for example over a time interval of 12 h, the bioreactor can replenish the amount of the organism taken from the inoculum in that the culturing of the organism in the inoculum continues under suitable conditions. In this way, at the end of the test series, there may again be sufficient biomass of the organism available in the inoculum within the bioreactor in order to enable taking of another sample (for example of the same size). It will be apparent that this process can be repeated several times in succession over the entire duration of a test sequence composed of multiple test series.

There are also conceivable applications in this context in which there are multiple different organisms present in the inoculum that are to be cultivated collectively by the apparatus in the culture medium. For example, this approach can be used to examine optimal culturing conditions for symbiotic pairs of organisms.

The amount of inoculum taken may, as mentioned, be below a particular maximal amount of, for example, 5% of the volume of the bioreactor system. For example, within 12 hours in which a test series is conducted, the controlled bioreactor can re-establish the originally present density of the organism (for example a cell density, or detected in an optically indirect manner as an optical density) in the inoculum, such that constant starting conditions again exist for the next test series.

If the cell density in the inoculum increases excessively, for example, the inoculum can be diluted in an automated manner, for example by means of the robot or else by means of a pump of the bioreactor, which can lower the density of the organism again. If, by contrast, there is a decrease in density (for example: cell density or optical density detected by sensors in the bioreactor), it is possible, especially by means of pumps in the bioreactor or with the aid of the robot, to feed in fresh media in order to again stimulate the growth of the inoculum. The use of a pipetting robot for adjustment of the biomass concentration of the organism in the inoculum is an option particularly when two or more different inocula are to be stored in the apparatus.

Moreover, the temperature of this separate bioreactor may also be/have been controlled, and it may be the case that the organism is continuously mixed and agitated (for example by means of a magnetic stirrer) in a nutrient solution of the inoculum. The effect of these precautions is that, in each test series of the test sequence conducted, the starting conditions will always be the same in relation to the quality and density/biomass concentration of the organism in the inoculum. Moreover, the cleanliness of the respective reaction vessel is also important for this purpose, which does of course have to be cleaned and disinfected in a complex manner before the start of the current test series (in an automated manner with the aid of the robot), as will be described in detail.

What is thus proposed is in particular that the apparatus, preferably the inoculum bioreactor itself, comprises measurement and control means with which a biomass concentration (especially a particular biomass concentration) can be kept constant in the inoculum bioreactor (especially the particular inoculum bioreactor).

In a preferred configuration, the inoculum bioreactor may comprise: at least one, preferably temperature-regulated, inoculum reaction vessel in which the inoculum is storable over several days, preferably over at least two weeks; and/or at least one inoculum adjustment means for adjustment of a composition of the inoculum, especially a culture medium of the inoculum. Such an inoculum adjustment means may especially be implemented by means of at least one pump for conveying a constituent of a culture medium of the inoculum into the inoculum reaction vessel and/or by means of at least one pump for conveying a portion of the inoculum out of the inoculum reaction vessel. The inoculum bioreactor may further comprise at least one, preferably optical, inoculum measurement device configured to measure a biomass/volume concentration of the organism to be cultured within the inoculum or within the inoculum reaction vessel. It will be apparent that it is advantageous for this purpose when the inoculum reaction vessel (just like the other reaction vessels of the apparatus) is configured to be transparent to a particular measurement wavelength, where this measurement wavelength is evaluated by the respective measurement device.

“Storing” of the inoculum may be understood here to mean that the inoculum is cultured continuously by the bioreactor in a suitable culture medium within the inoculum reaction vessel under suitable and uniform physiological conditions, in particular without any outside human involvement, i.e. in a fully automated manner. For this purpose, the inoculum bioreactor may be configured, especially by appropriate actuation of the closed-loop temperature control system described, the at least one adjustment means and evaluation by the measurement device, to keep a volume concentration of the organism constant within the inoculum (within a target interval). Suitable bioreactors that can be used as part of the apparatus in order to store the inoculum are already commercially available.

The inoculum bioreactor may additionally comprise a closed-loop controller configured to actuate at least one inoculum adjustment means depending on a measurement conducted with the at least one inoculum measurement device such that a volume concentration of the organism to be cultured can be kept within a defined interval within the inoculum. The interval thus defines the range in which the volume concentration of the organism may vary.

It is also very particularly advantageous when the robot is configured to clean the reaction vessels. This can be conducted especially after performance of a test series, wherein the organism has been cultivated in different compositions of the culture medium in the individual reaction vessels during the test series. The cleaning can preferably be accomplished by rinsing and/or by disinfecting, where the latter may especially be accomplished by rinsing with a disinfectant. It is particularly preferable here when the robot follows a fixed schedule in the cleaning and disinfecting of the reaction vessels, i.e., for example, a particular pipetting protocol comprising two or more purge steps and a wait interval in which the disinfectant (for example an alcohol solution) can act on the respective reaction vessel.

It may be the case, for example, that the reaction vessels are purged, cleaned and disinfected by the robot in a fixed time interval of, for example, 12 hours, in particular after each execution of a growth phase of the organism during a 12 h test series and a final optical measurement of the respective reaction vessel to ascertain the respective process response.

For example, after optical measurement of the N reaction vessels and subsequent computer-implemented assessment of a test series, each individual one of the reaction vessels (for example 48 micro-wells of a microtitre plate) may be rinsed repeatedly with sterile water by the pipetting robot (injection of water, clearing by suction; then start again) and subsequently disinfected by the pipetting robot with an alcohol solution that acts for a certain time before another rinsing operation. In this context, it is possible to resort to the pipetting protocol previously known in the prior art. As a result, it is possible to repeatedly use the reaction vessels, which may be implemented in particular by means of a common test platform, for example a well plate. As a result, at least during a test sequence, the test platform does not have to be exchanged, not even when it is actually configured as a disposable article. This procedure additionally eliminates the risk that residues from a preceding test series remain in the respective reaction vessel, as a result of which the test result of the subsequent test series would no longer be directly attributable to the new composition of the culture medium used therein, and the learning of the optimal composition/optimal contents of the different organisms by the Al would be made more difficult or even become impossible.

The said at least one process measurement device may preferably be configured (especially in each case) as an optical process measurement device. Moreover, it is preferable when this implements an optical transmittance measurement and/or an optical backscatter measurement.

The invention especially proposes that the process measurement device is configured to measure a respective change in the biomass of the organism (as the generated process response) in the respective reaction vessel.

Alternatively or else additionally to the detection of the change in biomass as a possible process response generated by the organism, the process measurement device may alternatively be configured to measure changes in at least one measurement parameter of the culture medium (for example pH and/or dissolved oxygen (DO)) which are caused by the organism in the respective reaction vessel. This approach may be an option, for example, when the reaction vessels, as described, are configured in a bioreactor and/or when, for example, it is not possible to directly detect the biomass concentration.

Specifically an optical backscatter measurement in which, for example, the intensity of light backscattered by the culture medium and the organism present therein is measured is of excellent suitability for obtaining an approximately linear measurement signal over a wide measurement range that permits conclusion of the current volume concentration of the organism within the reaction vessel/culture medium. The controller is able to use this to determine the growth of the organism, especially the progression over time of growth (observed, for example, over the period of time of a test series) of biomass in the reaction vessel as process response.

Depending on the apparatus configuration, it may have, for example, just one single process measurement device with which each of the reaction vessels can be measured, especially sequentially one after another. For example, when the reaction vessels are configured as cavities of a microtitre plate, the latter can be moved, for example by means of a step motor, across the process measurement device such that the process measurement device can optically measure each individual one of the cavities without having to move the process measurement device itself. Alternatively, however, it is of course equally possible not to move the reaction vessels and instead to move the process measurement device to a respective measurement point relative to the reaction vessels.

Alternatively thereto, it is also possible for each of the reaction vessels to have a dedicated process measurement device with which a respective process response generated by the organism in the respective reaction vessel can then be measured, in particular simultaneously and hence more quickly.

The detection of the respective process response can be effected once during a test series conducted, for example at the end thereof, or else quasi-continuously, i.e., for example, at particular time intervals during the duration of the respective test series. It is thus possible under some circumstances with the at least one process measurement device especially to detect/record a progression over time of the respective process response which is generated by the organism in the respective reaction vessel. This is because such a progression of the process response over time can be assessed by the controller against a complex objective that may include, for example, particular target marks over time.

It is likewise possible, as elucidated in detail below, to vary the composition of the culture medium during a test series conducted, for instance by quasi-continuous addition of at least one component of the culture medium, and/or to vary the ratio of the two organisms stepwise during a test series conducted.

The object mentioned is achieved in accordance with the invention, in a process of the type specified at the outset, by the features of the independent method claim. In particular, the invention thus proposes, for achievement of the object, in a process of the type described at the outset, that a number of N spatially separate reaction vessels are each filled with a fixed composition of the culture medium (to be optimized), which can preferably be effected in an automated manner with the aid of a robot; that, in addition, the N reaction vessels are inoculated, preferably with the robot (which may be configured as described above), with an inoculum comprising the respective organism (i.e. in particular one of the at least two organisms) (it will be apparent that, in the case of co-culturing of at least two organisms, at least two such individual inoculations of the respective reaction vessel may be undertaken); that a test series of N culturing tests that proceed in parallel, i.e. preferably simultaneously, in the N reaction vessels with the organism/the at least two organisms is subsequently conducted, where the respective organism, especially the at least two organisms together, in each of the N reaction vessels executes/collectively execute a respective biological process. Moreover, a respective process response and/or a respective process state that the organism/the at least two organisms generate(s) in the respective reaction vessel is measured with at least one process measurement device; this can especially be accomplished during and/or at the end of the test series, as already elucidated above with reference to the apparatus.

Finally, the N measured process responses and/or N measured process states may be assessed in a computer-implemented manner, preferably using artificial intelligence, in each case with reference to an objective (where the objective is preferably applicable to all the biological processes), and, on the basis of these N assessments, in a computer-implemented manner, a respective composition of the culture medium which is to be made up in the respective reaction vessel in a subsequent test series and/or which is to be varied during a subsequent respective culturing test is defined by means of an instruction in a computer-implemented manner.

If, by contrast, the said ratio between the at least two organisms is to be optimized, it is possible as an alternative or else in addition to the latter feature, in the method, that, on the basis of these N assessments, in a computer-implemented manner, the ratio of the at least two different organisms which is to be made up in the respective reaction vessel in a subsequent test series and/or which is to be varied during a subsequent respective culturing test is defined by means of an instruction in a computer-implemented manner.

In this method, it is preferably possible to use an apparatus configured in accordance with the invention as described above or according to one of the claims directed to such an apparatus in order to execute the above-elucidated individual method steps, especially in each case without human involvement and hence in an automated manner. The method can thus be executed autonomously by the apparatus; all that have to be provided are the necessary media and the inoculum/inocula. Conversely, an apparatus of the invention may of course be configured to execute a method as described and/or claimed herein, especially in such a way that the apparatus executes the method autonomously.

For example, in the method, it may be the case that the robot executes the instruction at the start of the subsequent test series by establishing the composition of the culture medium defined in each case by the instruction in each of the N reaction vessels and/or establishing the ratio of the at least two different organisms defined in each case by the instruction (for instance by pipetting suitable volumes of at least two different inocula into the respective reaction vessel).

In the method, the above-described test series may be conducted iteratively as a sequence of two or more test series that are successive in time. It is preferable here when the composition of the culture medium is brought stepwise to an optimum on the basis of the N assessments made in each of the individual test series. At the same time, simultaneously or else separately therefrom, it is also possible to optimize the described ratio of the at least two different organisms in a stepwise manner.

For example, with the aid of the method, it is possible to ascertain an optimized composition of the culture medium step by step. As a result, it is thus possible for at least one of the N measured process responses to move closer to the objective. In this case, the composition of the culture medium with which this process response has been generated may be regarded as the optimized composition. It will be apparent that this concept can be utilized in an analogous manner in order to optimize said ratio of the at least two different organisms (either additionally or alternatively to the optimization of the composition of the culture medium).

For example, it may also be the case that, during a running culturing test, the respective composition of the culture medium (with which the respective reaction vessel is filled) and/or the respective ratio of the at least two (different) organisms (which are being cultivated at any time within one of the reaction vessels) in the respective reaction vessel is/are varied. Such a change can thus especially be undertaken during a running culturing test. Variation of said ratio during the test may also be undertaken in addition to a change in the composition of the culture medium and/or in said ratio, in each case at the start of a new test series, i.e. in each case at the start of a respective culturing test. This can be accomplished, for example, in that (especially with the aid of the robot mentioned) individual components of the culture medium and/or at least one inoculum of one of the at least two organisms mentioned are added to the respective reaction vessel during a running culturing test. In this case, it is also possible that a certain amount of the culture medium together with the organism(s) present therein has been removed beforehand, especially in order thus to avoid an overflow of the reaction vessel and/or in order thus to more easily adjust the ratio. With such configurations, it is thus especially possible to conduct fed batch tests in an automated manner.

The said instruction can thus especially determine how the composition and/or the said ratio of the at least two organisms should be varied during a respective culturing test running at any time in the respective reaction vessel. This approach is especially suitable for the optimization of fed batch processes; this means batch processes that are processed as a “stack”, i.e. successively one after another, for instance by stepwise or continuous feeding-in of reactants. In other words, under some circumstances, the process regime in a method of the invention may be configured such that the composition of the culture medium is varied stepwise or even continuously during a culturing test.

In the method, it may further be the case that the reaction vessels, at the end of the respective test series, are cleaned and/or disinfected, preferably with the aid of the robot. This can especially be accomplished by rinsing out the culture medium and subsequently treating the respective reaction vessel with a disinfectant.

Finally, in the method, it may also be the case that the inoculum with which the N reaction vessels are newly inoculated at the start of each test series are drawn from a separate inoculum bioreactor (i.e. arranged separately from the reaction vessels). This withdrawal can preferably be accomplished with the aid of the robot described above. In addition, the inoculum bioreactor may of course be configured as described above. It is also preferable that the incubation conditions of the inoculum and/or a volume concentration of the organism in the inoculum are kept constant in an automated manner by the inoculum bioreactor over an entire duration of a sequence of test series conducted.

### BRIEF DESCRIPTION OF THE DRAWINGS

The invention is now described in detail by working examples, but is not limited to these. Further developments of the invention can be obtained in an obvious manner from the description of a preferred working example which follows in conjunction with the preceding general description, the claims and the corresponding drawings.

In the description of various embodiments of the invention that follows, elements of corresponding function are given corresponding reference numerals, even if they differ in configuration or shape.

The figures show:

FIG. 1 a schematic view of a first apparatus of the invention, which is envisaged and configured to ascertain an optimized composition of a culture medium in which a biological organism is to be cultured;

FIG. 2 a schematic view of a second possible configuration of an apparatus of the invention, with which a ratio of two microorganisms being co-cultured in a culture medium can be optimized autonomously.

### DETAILED DESCRIPTION

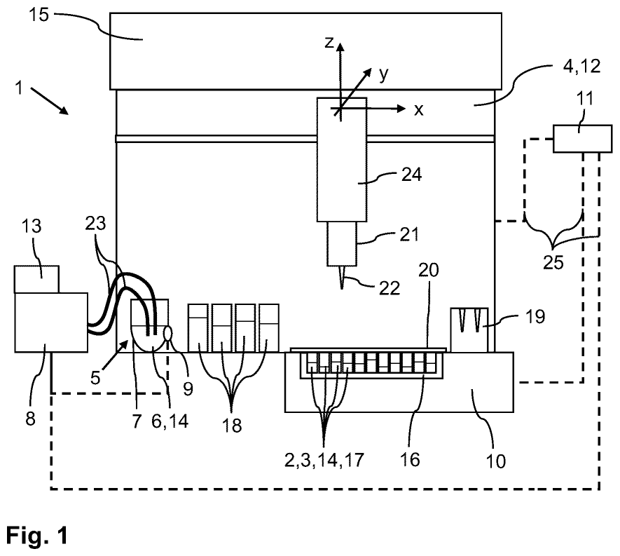

FIG. 1 shows an apparatus of the invention, labelled 1 as a whole, which has a microtitre plate 16 having numerous microtitre cavities 17 that are arranged in an array and each serve as a reaction vessel 2. With the aid of a pipetting robot 12 of the apparatus 1, which is controlled by means of a controller 11, a pipette 21 can be moved in the three spatial directions x, y, z, such that, with the aid of the pipetting robot 12, different compositions of a culture medium 3 can be established in the respective reaction vessels 2. For this purpose, several reservoir vessels 18 are provided, which each serve to store a particular media component, where individual amounts of the respective medium can be withdrawn in an automated manner with the pipette 21. The pipetting robot 12 can thus, on the basis of an instruction that it receives from the controller 11, withdraw different volumes of the respective medium from the respective reservoir vessel 18 and dispense them into the respective reaction vessel 2, in order thus to establish different compositions of the culture medium 3 in the individual reaction vessels 2.

The apparatus 1 further comprises a separate inoculum bioreactor 5 in which an inoculum 6 of an organism 14 to be cultured is stored under constant conditions. For this purpose, the inoculum bioreactor 5 has a measurement device 9 with which a volume concentration of the organism 14 can be constantly detected by measurement within an inoculum reaction vessel 7 of the bioreactor 5. Depending on these measurements, a closed-loop controller 13 actuates an adjustment means 8 with which the composition of the inoculum 6, more specifically of the culture medium of the inoculum 6 in which the organism 14 is constantly cultivated, can be adjusted. In the example shown in FIG. 1, the adjustment means 8 is implemented in the form of several pumps with which constituents of the culture medium of the inoculum 6 can be replenished into the inoculum reaction vessel 7 via the illustrated hoses 23. The controller 11 may also have a controlling and regulating effect via the control conduits 25 on the closed-loop controller 13 of the inoculum bioreactor 5, for instance in order to vary the biomass concentration of the inoculum 6.

In addition, the apparatus 1 comprises a filter module 15 in the form of an HEPA module with which a sterile gas atmosphere can be provided above the reaction vessels 2, which serves to prevent contamination of the individual reaction vessels 2.

With the apparatus 1, it is also possible, for a particular organism 14 which is stored in the inoculum 6 at constant biomass concentration, to optimize the composition of the culture medium 3 in which the organism 14 is to be cultured in the N reaction vessels 2 of the microtitre plate 16. It will be apparent that the culture medium 3 may differ here from that of the inoculum 6.

In order to optimize the composition, the apparatus 1 autonomously conducts at least one test series consisting of N culturing tests. For this purpose, the pipetting robot 4, 12 in each of the N reaction vessels 2 establishes a respective composition of the culture medium 3 (as elucidated above), where the initial composition may at first be chosen randomly, but is defined automatically by the controller 11. Subsequently, the robot 4 inoculates each of the N reaction vessels 2 with a certain amount of the inoculum 6 that the robot 4 withdraws from the inoculum bioreactor 5. In order to avoid cross-contamination, the robot 4 goes to a cleaning station 19 between the individual inoculations, where it cleans its pipette tip 22. The pipette tip 22 which is thus kept sterile can thus always release a uniform quality of the inoculum 6 into the respective reaction vessel 2.

Once all the reaction vessels 2 have been inoculated, the apparatus 1 performs culturing of the organism 14 under suitable conditions over a defined period of time. For example, the conditions (temperature, pressure, atmosphere) may be chosen such that a desired growth of the organism 14 and hence an increase in biomass in the respective reaction vessel 2 is to be expected. At the end of this growth phase, the apparatus 1 autonomously measures the growth of biomass in the organism 14 in the respective reaction vessel 2 as a process response with the aid of a process measurement device 10. This process measurement device 10 is preferably of optical configuration and may, for example, detect the growth of biomass in the respective reaction vessel 2 by means of optical backscatter measurement or measurement of the respective optical density of the culture medium present in the respective reaction vessel 2. This is achieved in a particularly simple manner, for example, when the optical process measurement device 10 has a static measurement head and the controller 11, by means of corresponding control signals and actuation, moves the microtitre plate 16 relative to the static measurement head, such that the optical process measurement device 10 can optically measure each of the N reaction vessels 2 individually (and sequentially one after another). Alternatively, however, it is also possible, for example, to use two or more process measurement devices 10 in order to conduct a dedicated measurement for each of the N reaction vessels 2 (in this case, it is then possible to perform several measurements simultaneously).

The controller 11 then processes the N measurements that have been measured (as process responses or process states) in the course of the above-described first test series for the N reaction vessels 2 by means of the process measurement device 10, and assesses these using an objective, for example a particular desired increase in biomass concentration per unit time, i.e. a particular growth rate. It may be the case here, for example, that particular compositions of the culture medium 3 that have been produced in some of the N reaction vessels 2 by the robot 4 lead to an undesirable low growth rate, and others to an undesirable high growth rate. The controller then processes these N assessments and then gives the robot 4 an instruction as to which new N compositions of the culture medium 3 the robot 4 should establish in the respective reaction vessels among the N reaction vessels 2 in a subsequent test series.

In order again to create the same starting conditions for the subsequent new test series from N culturing tests, the pipetting robot 12 first cleans all of the N reaction vessels 2 by rinsing them out and then treating them with a disinfectant. The reaction vessels 2 that have thus been cleaned and sterilized are then available for refilling with the culture medium 3.

In the second test series that then follows, the robot 4, 12 then again fills each of the N reaction vessels 2 with a respective composition of the culture medium 3 defined by the described instruction from the controller 11. Subsequently, the robot 4 inoculates all N reaction vessels 2 again with a respective sample of the inoculum 6, such that a new growth phase of the same organism 14 in the respective reaction vessel 2 can proceed subsequently. At the end of this growth phase, the duration of which can also be varied by the controller 11, the apparatus 1 again autonomously measures the respective growth of biomass concentration in the respective reaction vessel 2 with the aid of the process measurement device 10, and the controller 11 again makes N assessments of these process responses measured at the end of the second test series. Subsequently, the controller 11 again issues an instruction, which allows definition of another adjustment of the respective composition of the culture medium 3 in the respective reaction vessel 2, in that case for the third subsequent test series.

In this way, i.e. by repeated sequential execution of such test series each of N culturing tests that proceed in parallel and simultaneously, the controller 11 is able to learn bit by bit from the respective assessments how the composition of the culture medium 3 can be optimized to the organism 14 such that the desired objective for the growth rate can be achieved. For this purpose, the controller 11 has artificial intelligence which is implemented as a neural network and implements a machine learning method. This means that the controller 11 is able to learn the optimized composition of the culture medium 3 from the respectively measured process responses or process states (both approaches are possible) autonomously—i.e. without human involvement. At the end of a sequence of multiple test series conducted (each of which comprises N culturing tests), the controller can then output the optimized composition of the culture medium 3 found (preferably in digital form). It should also be mentioned that the controller, on the basis of the measured process responses and/or states, can also issue an instruction, for example, as to how the respective composition of the respective culture medium should be varied in each case during a test series of N tests that is currently running, for instance when a fed batch process is being executed by the apparatus 1 in the respective reaction vessel.

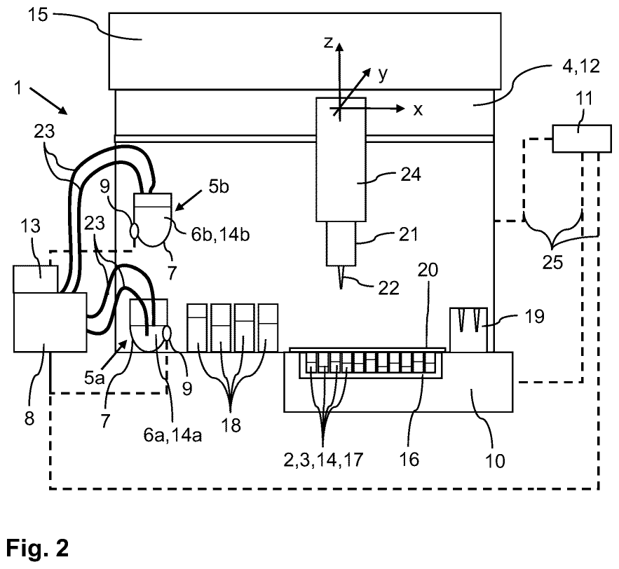

FIG. 2 shows a further possible configuration example of an inventive apparatus 1, with which, as already in the example of FIG. 1, a method of the invention can be executed in a computer-implemented manner. By contrast with the apparatus 1 according to FIG. 1, that in FIG. 2 has a second inoculum bioreactor 5b. While a first organism 14a is thus stored in the first inoculum bioreaction vessel 7a of the first inoculum bioreactor 5a, the second inoculum 6b in the second inoculum bioreaction vessel 7b provides a second organism 14b. Both inoculum bioreactors 5a and 5b are monitored and regulated by the common closed-loop controller 13.

It is thus possible with the pipetting robot 12, especially in the case of an initially unchanged composition of the culture medium 3, to establish different ratios of amount between the two organisms 14a and 14b in the respective reaction vessel 2 of the microtitre plate 16, where the two organisms 14a and 14b are each cultured together in the culture medium 3 in the respective reaction vessel 2.

In the configuration of FIG. 2 as well, it is thus possible to conduct N culturing tests each in parallel and simultaneously with the apparatus 1, although it is not obligatory here to change the respective composition of the culture medium 3 in the respective reaction vessel 2; instead, the controller 11 may first attempt, for example, to optimize the said ratio of the two organisms 14a and 14b, for instance with regard to a desired process state. Such a process state may consist, for example, in co-culturing the two organisms 14a and 14b together for as long as possible. Once the controller 11 has found an optimal ratio between the two organisms 14a and 14b over several test series, it is then able to additionally learn autonomously how the composition of the culture medium 3 can possibly be further optimized for the ratio found. Of course, other optimization approaches would also be possible with the apparatus 1 of FIG. 2: for example, the controller 11 could first attempt to optimize a composition of the culture medium 3 for one of the two organisms 14a and 14b, and then attempt to culture both the organisms 14a and 14b in a suitable mixing ratio in this optimized composition found.

In summary, in order to accelerate the determination of optimal conditions for culturing of a particular biological organism 14, an apparatus 1 and a corresponding method are proposed, with which, with the aid of a computer and autonomously, i.e. without any human involvement, an optimized composition of a culture medium 3 and optionally additional optimized active factors, especially an optimized ratio of (at least) two organisms (14a, 14b) that are to be cultivated together, can be ascertained. For this purpose, the biological organism 14 to be examined, which is kept alive and hence ready for the experiments in the form of an inoculum 6 in a separate inoculum bioreactor 5 over the entire test duration, is introduced with the aid of a robot 4 into individual reaction vessels 2 in which fixed specific compositions of the culture medium 3 are present and/or a particular proportion of a second organism 14 is already present. Subsequently, the organism 14 is cultured in the respective culture medium 3, i.e. within the respective reaction vessel 2, and a process response being generated by the biological organism 14 or a process state in which it currently exists is measured with the aid of a process measurement device 10. Finally, the apparatus 1 assesses, using an objective, whether the defined culturing conditions in the respective reaction vessel 2 have led to a desired process response/process state.

Depending on the result of these N assessments in total, where the respective assessment is conducted in a computer-implemented manner for each of the N reaction vessels 2, it is then possible for a controller 11 of the apparatus 1 to decide how the composition of the culture medium 3 and, if appropriate, further action parameters are to be defined and adjusted or varied in a subsequent test series. For this purpose, in this subsequent test series, all the reaction vessels 2 are then newly filled with the culture medium 3, and also newly inoculated in an automated manner with the organism 14 to be examined/the organisms 14a, 14b to be cultured. By this approach, it is possible in a very short time to determine, in an automated manner, the optimized composition of the culture medium 3 for a specific biological organism 14 and/or an optimized ratio of at least two organisms 14a, 14b to be cultured together.

### LIST OF REFERENCE NUMERALS

1 apparatus
2 reaction vessel
3 culture medium
4 robot
5 inoculum bioreactor (may include 6 and 7)
6 inoculum (stored in 7)
7 inoculum reaction vessel
8 inoculum adjustment means (for adjustment of a composition of a culture medium of 6)
9 inoculum measurement device process measurement device
11 controller
12 pipetting robot
13 closed-loop controller (for control of the incubation conditions in 5)
14 organism
15 filter module (e.g. HEPA module; for avoidance of contamination)
16 microtitre plate
17 microtitre cavity
18 reservoir vessel (for storage of a media component)
19 cleaning station for pipette tip
20 sterile barrier
21 pipette
22 pipette tip
23 hose
24 pipetting arm (movable, for movement of 21/22)
25 control conduits (of 11, for readout and/or actuation of 4/12/10/8)

1. An apparatus (1) for computer-implemented determination of an optimized composition of a culture medium (3) and/or of an optimized ratio of at least two organisms (14a, 14b), the apparatus (1) comprising:
a number of N spatially separate reaction vessels (2) fillable with N compositions of the culture medium (3) in order to culture in each of the N compositions, in the course of a test series composed of N culturing tests, at least one biological organism (14) that executes a biological process,
a robot (4) configured to produce the respective composition of the culture medium (3) in the respective reaction vessel (2), and
at least one process measurement device (10) configured to measure a respective process response and/or a process state which is generated within the respective reaction vessel (2) by the at least one organism (14) during the test series,
a controller (11) configured to
assess the total of N process responses and/or N process states that have been measured by the at least one process measurement device (10) during the respective test series, in each case in a computer-implemented manner with reference to a common objective which is applicable to all of the processes that proceed in the reaction vessels (2) in parallel, and based on those N assessments, in a computer-implemented manner, to give the robot (4) one or more of the following in the form of an instruction:
the respective composition of the culture medium (3), or
a ratio of at least two different organisms (14a, 14b)
which is to be established in the respective reaction vessel (2) in a subsequent test series.

2. The apparatus (1) according to claim 1, wherein the robot (4) is configured as a pipetting robot (12), and includes
a movable pipetting arm (24) configured to be positioned over each one of the N reaction vessels (2) in order to at least one of dispense individual components of the culture medium (3) into the respective reaction vessel (2) or suction the respective culture medium (3) out of the respective reaction vessel (3).

3. The apparatus (1) according to claim 1, wherein the controller (11) is configured to control the robot (4) with using artificial intelligence, such that the apparatus (1) learns independently from at least one of the measured process responses or process states over a course of multiple successive test series how the robot (4) can establish an optimized composition of the culture medium (3).

4. The apparatus (1) according to claim 1, wherein the controller (11) has artificial intelligence which automatically learns an optimized composition of the culture medium (3) from the respectively measured process responses, and outputs the optimized composition of the culture medium (3) at an end of a sequence of multiple test series conducted with the apparatus (1).

5. The apparatus (1) according to claim 1, further comprising at least one inoculum bioreactor (5) in which an inoculum of the organism (14) to be cultured is at least one of adapted to be stored or is stored, and
the robot (4) is configured to take a sample of the inoculum from the inoculum bioreactor (5) and to inoculate the respective reaction vessel (2) with said sample.

6. The apparatus (1) according to claim 5, wherein the inoculum bioreactor (5) comprises at least one of:
at least one inoculum reaction vessel (7), in which the inoculum (6) is storable over several days,
at least one inoculum adjustment device (8) for adjusting a composition of the inoculum (6), including at least one of a pump for conveying a constituent of a culture medium of the inoculum (6) into the inoculum reaction vessel (7) or a pump for conveying a portion of the inoculum (6) out of the inoculum reaction vessel (7), or
at least one inoculum measurement device (9) configured to measure a volume concentration of the organism (14) to be cultured within the inoculum (6).

7. The apparatus (1) according to the claim 6, further comprising a closed-loop controller (13) of the inoculum bioreactor (5) that is configured to actuate the at least one inoculum adjustment device (8) depending on a measurement conducted with the at least one inoculum measurement device (9) such that a volume concentration of the organism (14) to be cultured within the inoculum (6) is kept within a defined interval.

8. The apparatus (1) according to claim 1, wherein the robot (4) is configured to clean the reaction vessels (2) by at least one of rinsing out or disinfection, and the robot (3) follows a fixed schedule in the cleaning and disinfecting of the reaction vessels (2).

9. The apparatus (1) according to claim 1, wherein the at least one process measurement device (10) is an optical process measurement device (10), and the optical process measurement device (10) implements at least one of an optical transmittance measurement or an optical backscatter measurement.

10. The apparatus (1) according to claim 1, wherein the apparatus (1) has just one said process measurement device (10) with which each of the reaction vessels (2) is measurable, or each of the reaction vessels (2) has a dedicated process measurement device (10) with which a respective process response generated by the organism in the respective reaction vessel (2) is measurable.

11. A method of computer-implemented determination of an optimized composition of a culture medium (3) in which at least one organism (14) is to be cultured, and/or an optimized ratio of at least two organisms (14a, 14b) that are to be cultured collectively in a culture medium, the method comprising:
filling each of a number of N spatially separate reaction vessels (2) with a fixed composition of the culture medium (3),
inoculating the N reaction vessels (2) with an inoculum (6) comprising the respective organism (14),
conducting a test series of N culturing tests that proceed in parallel in the N reaction vessels (2) with the organism (14) or the at least two organisms (14a, 14b), with the respective organism (14) in each of the N reaction vessels (2) executing a respective biological process,
measuring at least one of a respective process response or a respective process state that the organism (14) generates in the respective reaction vessel (2) with at least one process measurement device (10),
assessing the N measured process responses and/or process states in a computer-implemented manner, in each case with reference to an objective, and
based on these N assessments that are made in the computer-implemented manner, defining at least one of a) a respective composition of the culture medium (3) or b) a ratio of the at least two different organisms (14), which is to be made up in the respective reaction vessel (2) in a subsequent test series and/or which is to be varied during a subsequent respective culturing test, by an instruction in a computer-implemented manner.

12. The method according to claim 11, further comprising at least one of
the robot, at a start of the subsequent test series, executing the instruction by establishing, in each of the N reaction vessels (2), the respective composition of the culture medium (3) defined by the at least one of the instruction or the respective ratio of the at least two organisms defined by the instruction, or
while a culturing test is in progress, varying the respective composition of at least one of the culture medium or the respective ratio of the at least two different organisms (14) in the respective reaction vessel.

13. The method according to claim 11, further comprising conducting the test series iteratively as a sequence of multiple test series that follow one after another, wherein at least one of the composition of the culture medium (3) or said ratio is brought stepwise to an optimum based on the N assessments respectively made in the individual test series, such that at least one of the N measured process responses moves closer to the objective.

14. The method according to claim 11, further comprising at least one of cleaning or disinfecting the reaction vessels (2) at an end of the respective test series, by rinsing out the culture medium (3) and treating the respective reaction vessel (2) with a disinfectant.

15. The method according to claim 11, further comprising drawing the inoculum (6) with which the N reaction vessels (2) are inoculated again at a start of each test series from a separate inoculum bioreactor (5), and
keeping incubation at least one of conditions of the inoculum (6) or a volume concentration of the organism (14) in the inoculum (4) constant in an automated manner by the inoculum bioreactor (5) over an entire duration of a sequence of the test series.

16. The apparatus (1) according to claim 1, further comprising a separate inoculum bioreactor (5) in which an inoculum (6) of the organism (14) to be cultivated is storable or is stored.
